(19) United States
(12) Patent Application Publication
(10) Pub. No.: US 2014/0099633 A1
(43) Pub. Date: Apr. 10, 2014

(54) DETECTION OF miRNA USING CAPILLARY ELECTROPHORESIS WITH LASER-INDUCED FLUORESCENCE DETECTION

(71) Applicant: KOREA INSTITUTE OF SCIENCE AND TECHNOLOGY, Seoul (KR)

(72) Inventors: Eunmi BAN, Seoul (KR); Dong-Kyu Chae, Seoul (KR); Eun Joo Song, Seoul (KR)

(73) Assignee: Korea Institute of Science and Technology, Seoul (KR)

(21) Appl. No.: 13/682,941

(22) Filed: Nov. 21, 2012

(30) Foreign Application Priority Data
Oct. 4, 2012 (KR) 10-2012-0110113

(51) Int. Cl. C12Q 1/68

(52) U.S. Cl. CPC C12Q 1/6883
USPC 435/6.11; 436/501

(57) ABSTRACT

Disclosed is a method for detecting a miRNA present in a sample in trace amounts and a kit for detecting the same. According to the present invention, the miRNA present in the sample in trace amounts can be quantitatively analyzed in short time. The detection method of the present invention may be used for fast diagnosis of various diseases wherein miRNAs are involved, for example, cardiovascular diseases including myocardial infarction.

### CROSS-REFERENCE TO RELATED APPLICATIONS

This application claims priority under 35 U.S.C. §119 to Korean Patent Application No. 10-2012-0110113, filed on Oct. 4, 2012, in the Korean Intellectual Property Office, the disclosure of which is incorporated herein by reference in its entirety.

### BACKGROUND

(a) Technical Field

The present invention relates to a method for detecting a miRNA present in a sample in a trace amount and a kit for detecting the same.

(b) Background Art

A microRNA (miRNA) is a very short non-coding RNA consisting of 21-22 nucleotides on average. By regulating other genes via inhibition of translation of mRNA, it can control cellular differentiation, embryogenesis, metabolism and oncogenesis. It is thought that about 30% of the total genes in human genome are regulated by miRNAs. The miRNAs are generated through transcription of individual genes in the non-coding regions. The miRNA is transcribed from a pri-miRNA which is a precursor transcribed in the nucleus by RNA polymerase II. The pri-miRNA is cleaved by the RNase III enzyme called Drosha (dsRNA-specific ribonuclease) to produce a pre-miRNA having a hairpin loop structure. The hairpin loop of the pre-miRNA is exported out of the nucleus by the proteins exportin-5 and Ran-GTP, which serve as cofactors, and processed into a miRNA duplex about 22 nucleotides in length by the action of the RNase III enzyme Dicer and TRBP (transactivation-responsive RNA binding protein). The miRNA duplex binds with RISC (RNA-induced silencing complex) and cleaves mRNAs or regulates genes by preventing translation.

Various kinds of miRNAs and target genes regulated thereby may be useful in predicting the mechanisms of various diseases. Since abnormally increased or decreased miRNA expression is observed in various diseases such as cancer, diabetes and cardiovascular diseases, the miRNA is recognized as a biomarker for diagnosing and predicting of diseases.

In particular, there has been active research on miRNA as a biomarker for diagnosis and prediction of cardiovascular diseases. Since the cardiovascular diseases are a major cause of death worldwide and need quick treatment, detection of useful biomarkers for fast and accurate diagnosis is required. In particular, myocardial infarction needs to be diagnosed early for fast recovery. Considering that miRNAs are found in a variety of biological substances including not only cells but also serum, blood plasma, saliva, tears and urine and the concentration of specific miRNAs shows difference in patients and healthy people, the miRNAs may be used as biomarkers of various diseases including the cardiovascular diseases.

The miRNAs associated with cardiovascular diseases is present in trace amounts in blood. Accordingly, a selective and highly sensitive analysis method is necessary to detect them. At present, microarray techniques and reverse transcription polymerase chain reaction techniques are mainly used for miRNA detection. Although the microarray technique allows detection of many kinds of miRNAs, quantitativity is not so good. Although the reverse transcription polymerase chain reaction technique is an excellent quantitative analysis method, it is costly, is problematic in reproducibility and, above all, requires a very long analysis time of 3-4 hours or longer. Therefore, many researchers are striving to develop a method for detecting a miRNA in short time with superior quantitativity, reproducibility and resolution.

Throughout the specification, a number of publications and patent documents are referred to and cited. The disclosure of the cited publications and patent documents is incorporated herein by reference in its entirety to more clearly describe the state of the related art and the present invention.

### SUMMARY

The inventors of the present invention have made efforts to establish a highly sensitive analysis method capable of detecting a miRNA present in a trace amount in a cell in short time with high accuracy. As a result, they have confirmed that the miRNA present in a trace amount in a cell can be detected with high sensitivity by hybridizing the miRNA using a probe capable of specifically hybridizing them and a suitable hybridization buffer and analyzing them using a CE/LIF system.

The present invention is directed to providing a method for detecting a miRNA present in a trace amount in a cell.

The present invention is also directed to providing a kit for detecting a miRNA.

In an aspect, the present invention provides a method for detecting a-miRNA in a sample, including:

(a) extracting an RNA from a sample to be analyzed;

(b) hybridizing the extracted RNA with a single-stranded DNA labeled with a fluorescent material as a probe specific to a miRNA expected to be present in the sample in a trace amount in a hybridization buffer;

(c) separating and identifying a DNA-miRNA complex using a capillary electrophoresis with laser-induced fluorescence detector (CE-LIF); and

(d) identifying the presence of the miRNA in the sample by analyzing a peak of the DNA-miRNA complex.

Other features and aspects of the present invention will be apparent from the following detailed description, drawings and claims.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 schematically shows the generation of a miRNA in a cell and regulation of a gene mediated by the miRNA;

FIG. 2 shows a process of hybridizing a target miRNA with a single-stranded DNA specific to the target miRNA and labeled with a fluorescent material and identifying the presence of the target miRNA by detecting the peaks of an unhybridized DNA and a DNA-target miRNA complex by CE/LIF, for quantitative or qualitative analysis of the miRNA;

FIG. 3 shows a result of detecting a single-stranded DNA specific to miRNA-499 and labeled with a fluorescent material only and identifying the peak of the DNA by CE/LIF (FIG. 3A) and a result of hybridizing miRNA-499 with the DNA and identifying the peaks of an unhybridized DNA and a DNA-miRNA-499 complex by CE/LIF (FIG. 3B);

FIG. 4 shows that, when miRNA-499 is hybridized with a single-stranded DNA specific to miRNA-499 and labeled with a fluorescent material, the intensity of the peak of a DNA-miRNA-499 complex varies depending on hybridization buffers and the highest intensity is obtained when Tris-EDTA-NaCl (TEN) buffer is used; and

FIG. 5 shows a result of performing hybridization in the hybridization buffer giving the highest intensity (see FIG. 4) by adding a single-stranded DNA specific to miRNA-499 and labeled with a fluorescent material to total RNAs extracted from H9c2 cardiomyocytes with or without miRNA-499 added and identifying the peaks of an unhybridized DNA and a DNA-miRNA-499 complex by CE/LIF (FIG. 5A shows a result for unhybridized DNAs, FIG. 5B shows a result for adding 50 fM of miRNA-499 to H9c2 cells, and FIG. 5C shows a result of detecting endogenous miRNA-499 of H9c2 cells without adding miRNA-499).

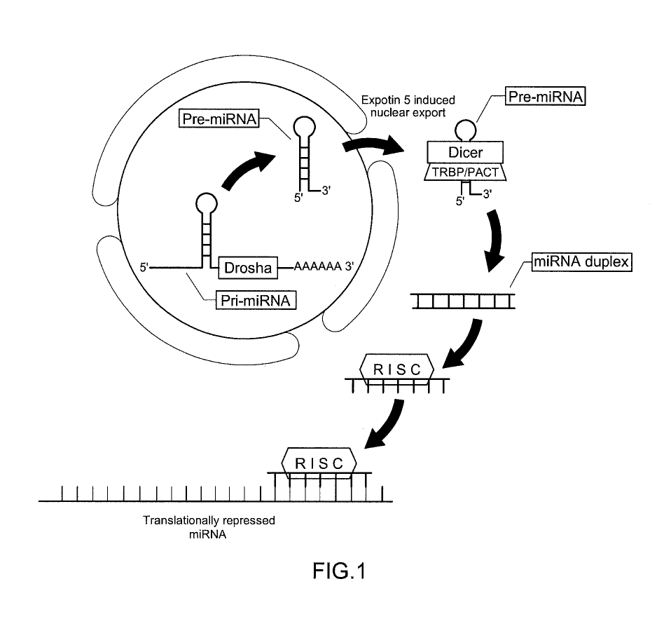

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The inventors of the present invention have made efforts to establish a highly sensitive analysis method capable of detecting a miRNA present in a trace amount in a cell in short time with high sensitivity and, as a result, they have confirmed that the miRNA is present in a trace amount in a cell can be detected with high sensitivity by hybridizing the miRNA using a probe capable of specifically hybridizing them and a suitable hybridization buffer and analyzing them using a capillary electrophoresis with laser-induced fluorescence detector (CE-LIF).

The miRNA may have an important role in predicting various diseases. That is to say, since abnormally increased or decreased miRNA expression is observed in various diseases such as cancer, diabetes and cardiovascular diseases, the miRNA may be used as a biomarker for diagnosing and predicting of diseases. However, since the miRNA is present in a trace amount in the body, a selective and highly sensitive analysis method is necessary for detection. At present, microarray techniques and reverse transcription polymerase chain reaction techniques are frequently used for miRNA detection. Although the microarray technique allows detection of many kinds of miRNAs, quantitativity is very poor. The reverse transcription polymerase chain reaction technique is problematic in that it is costly, has reproducibility problem and, above all, requires a very long analysis time of 3-4 hours or longer.

The detection method developed by the inventors of the present invention allows detection of the miRNA present in a sample in a trace amount for short time, specifically within 1 hour.

As used herein, the term “trace amount” refers to an amount in the level of femtomolar (fM) or less. Specifically, the “trace amount” may refer to an amount of 500 fM or less, more specifically 100 fM or less, most specifically 50 fM or less.

The extraction of total RNAs including the miRNA from the sample may be performed according to various methods known in the art. Specifically, TRIzol or Triton X-100 may be used for the extraction.

The detection method of the present invention may be used diagnose and predict various diseases, specifically to diagnose cardiovascular diseases including myocardial infarction.

Various miRNAs are known as biomarkers of cardiovascular diseases are (Wilson et al., Dynamic microRNA expression programs during cardiac differentiation of human embryonic stem cells: role for miR-499, Circ Cardiovasc Genet, 3(5): 426-35 (2010)). Accordingly, when the detection method of the present invention is used for detection of the biomarkers of cardiovascular diseases, the miRNA may be specifically miRNA-499, miRNA-1, miRNA-133 or miRNA-208, more specifically miRNA-499 of SEQ. ID. NO. 1, miRNA-1 of SEQ. ID. NO. 2, miRNA-133a of SEQ. ID. NO. 3 or miRNA-208 of SEQ. ID. NO. 4, most specifically miRNA-499 of SEQ. ID. NO. 1, as a biomarker of the cardiovascular diseases.

The miRNA may be present in trace amounts in various biological substances, including cells, serum, blood plasma, saliva, tears or urine. For example, when the miRNA is used as a biomarker of cardiovascular diseases, the cell may be a cardiomyocyte.

As used herein, the term “probe” refers to a natural occurring or modified monomer or linear oligomer including a deoxyribonucleotide or a ribonucleotide that can be hybridized with a specific nucleotide sequence. Specifically, the probe may be single-stranded for maximizing hybridization efficiency. And, specifically, the probe may be a deoxyribonucleotide.

As the probe, not only a sequence perfectly complementary to the sequence including the miRNA but also one substantially complementary thereto may be used, as long as the specific hybridization is not interfered with. When the detection method of the present invention uses miRNA-499, the probe used in the present invention may specifically include SEQ. ID. NO. 5.

The fluorescent material labeled at the probe may provide a signal allowing detection of the hybridization. The label may be attached to an oligonucleotide. The label that can be used in the present invention may be various fluorescent materials including fluorescein, phycoerythrin, rhodamine, lissamine, Cy3 and Cy5 (Pharmacia), and the like. Specifically, 6-carboxyfluorescein may be used. The labeling may be performed according to the methods commonly employed in the art. For example, nick translation, random priming (Multiprime DNA labeling systems booklet, Amersham (1989)) or kination (Maxam & Gilbert, Methods in Enzymology, 65: 499 (1986)) method may be used.

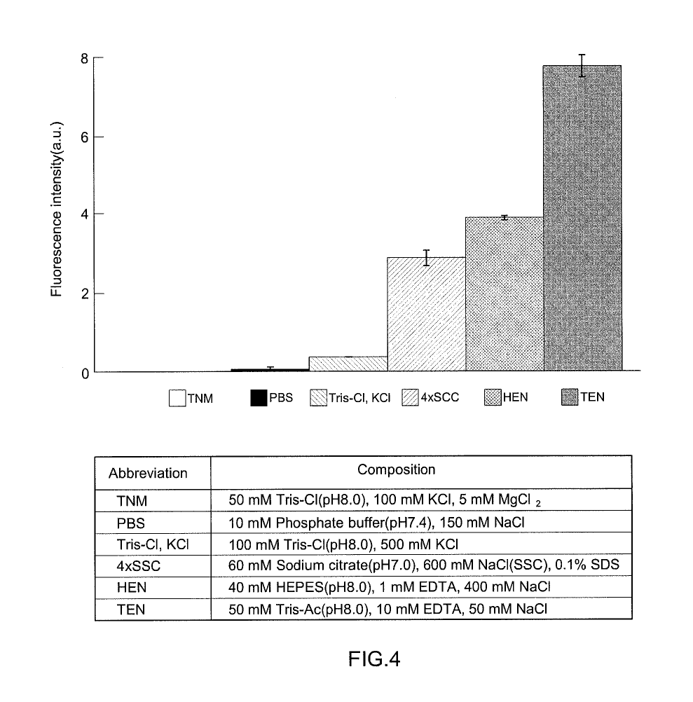

An optimal hybridization condition may be determined by referring to Molecular Cloning, A Laboratory Manual (Joseph Sambrook, et al., Cold Spring Harbor Laboratory Press, Cold Spring Harbor, N.Y. (2001)) and Nucleic Acid Hybridization, A Practical Approach (Haymes, B. D., et al., IRL Press, Washington, D.C. (1985)). A stringent condition for hybridization may be determined by adjusting temperature, ionic strength (buffer concentration) and the presence of other compounds such as organic solvents. The stringent condition may be determined differently depending on the hybridized sequences. Various kinds of hybridization buffers commonly used in the related art may be used in the present invention. A buffer exhibiting the highest hybridization efficiency may be selected. For example, TNM buffer, PBS buffer, Tris-Cl buffer, SSC buffer, HEN buffer or TEN buffer may be used as the hybridization buffer (see FIG. 4). Specifically, TEN buffer may be used.

The detection method of the present invention may further include determining a most suitable hybridization buffer by hybridizing the miRNA with a single-stranded DNA labeled with a fluorescent material as a probe specific for the miRNA in various hybridization buffers.

In an exemplary embodiment of the present invention, the capillary electrophoresis with laser-induced fluorescence detector (CE-LIF).

When the CE/LIF system is used, the hybridized DNA-miRNA complex may be separated using an uncoated capillary. Specifically, the capillary may have an inner diameter of 50-100 μm and a length of 20-60 cm, but is not limited thereto. The hybridized DNA-miRNA complex may be separated by applying a voltage of 10-20 kV, specifically 16 kV, into the capillary using Tris-borate buffer as a separation buffer.

The wavelengths of the LIF detector may be different depending on the fluorescent material to be detected. When 6-carboxyfluorescein is used as the fluorescent material, the excitation wavelength may be specifically in the range of 400-500 nm, most specifically 488 nm, and the emission wavelength may be specifically in the range of 500-600 nm, most specifically 520 nm.

In another aspect, the present invention provides a kit for detecting the miRNA.

In an exemplary embodiment of the present invention, the kit of the present invention is a kit for detecting the miRNA present in a trace amount of not more than 50 femtomolar, including: (a) a probe that can be specifically hybridized with the miRNA and a fluorescent material that can be labeled at the probe, or a probe labeled with a fluorescent material that can be specifically hybridized with the miRNA; (b) a hybridization buffer; and (c) a buffer for separation of a DNA-miRNA complex.

In an exemplary embodiment of the present invention, the miRNA may be one or more selected from the group consisting of miRNA-499, miRNA-1, miRNA-133 and miRNA-208.

In an exemplary embodiment of the present invention, the probe may be a probe of SEQ. ID. NO. 5.

In an exemplary embodiment of the present invention, the hybridization buffer may be one or more selected from the group consisting of TNM buffer, PBS buffer, Tris-Cl buffer, SSC buffer, HEN buffer and TEN buffer.

In an exemplary embodiment of the present invention, the separation buffer may be Tris-borate buffer.

In an exemplary embodiment of the present invention, the fluorescent material may be 6-carboxyfluorescein.

### EXAMPLES

The present invention will be described in more detail through examples. The following examples are for illustrative purposes only and it will be apparent to those skilled in the art not that the scope of this invention is not limited by the examples.

### Example 1

### Identification of Peaks of Unhybridized DNA and DNA-miRNA Complex by Capillary Electrophoresis

Four different miRNAs (miRNA-499 (5′-UUAAGACUUGCAGUGAUGUUU-3′, SEQ. ID. NO. 1), miRNA-1 (5′-UGGAAUGUAAAGAAGUGUGUAU-3′, SEQ. ID. NO. 2), miRNA-133a (5′-UUUGGUCCCCUUCAACCAGCUG-3′, SEQ. ID. NO. 3) and miRNA-208 (5′-AUAAGACGAGCAAAAAGC-3′, SEQ. ID. NO. 4)) were selected as biomarkers of cardiovascular diseases. Among them, miRNA-499 was used for experiment.

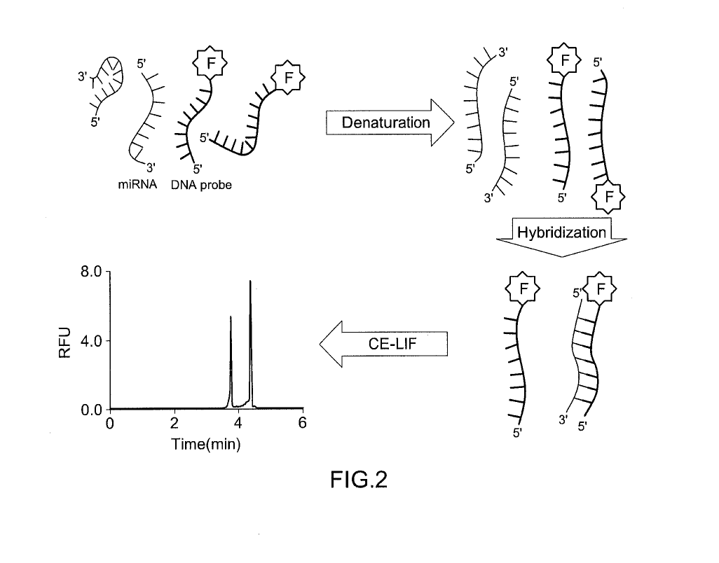

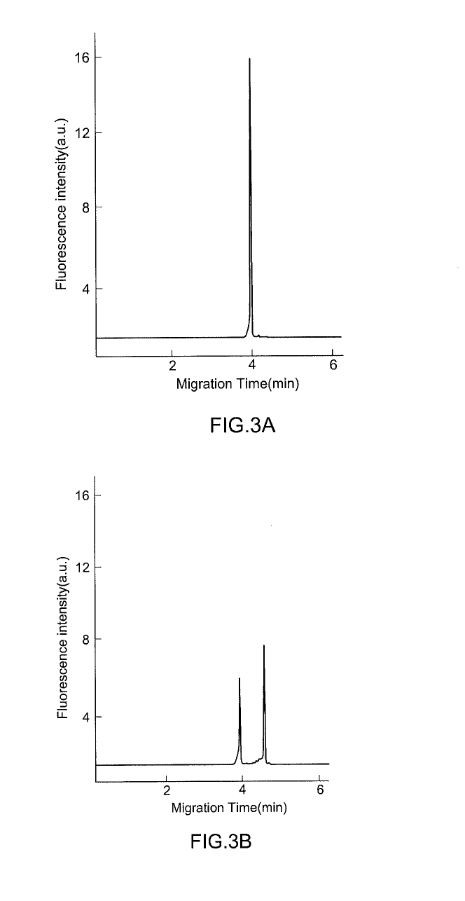

As described in FIG. 2, a DNA probe specific to miRNA-499 (5′-AAACATCACTGC AAGTCTTAA-3′, SEQ. ID. NO. 5) labeled with a fluorescent material, i.e., 5′-carboxyfluorescein phosphoramidite (6-FAM), and miRNA-499 were hybridized at 40° C. for 15 minutes in various hybridization buffers after preincubation at 95° C. for 5 minutes and then analyzed using a capillary electrophoresis with laser-induced fluorescence detector (CE-LIF). FIGS. 3A and B show the peaks of an unhybridized DNA and a DNA-miRNA-499 complex. The CE system was PA 800 plus CE system (Beckman Coulter, Fullerton, Calif., USA) and the LIF detector was Beckman P/ACE System Laser Module 488 with excitation and emission wavelengths of 488 nm and 520 nm, respectively. The DNA-miRNA-499 complex was separated in 100 mM Tris-borate buffer (×10.0) by applying a voltage of 16 kV into an uncoated capillary (Beckman Coulter) having an inner diameter of 75 μm and a length of 30 cm. Sample injection was carried out at 0.5 psi for 5 seconds.

### Example 2

### Determination of Optimal Hybridization Buffer

A DNA probe specific to miRNA-499 labeled with 6-FAM and miRNA-499 were hybridized at 40° C. for 15 minutes in various hybridization buffers after preincubation at 95° C. for 5 minutes and then analyzed using a capillary electrophoresis with laser-induced fluorescence detector (CE-LIF) (see FIG. 4). The analysis condition was the same as in Example 1. It was found out that the peak intensity of a DNA-miRNA complex varies greatly depending on hybridization buffers and that the highest intensity is obtained when TEN buffer is used.

### Example 3

### Detection of miRNA in Cardiomyocytes

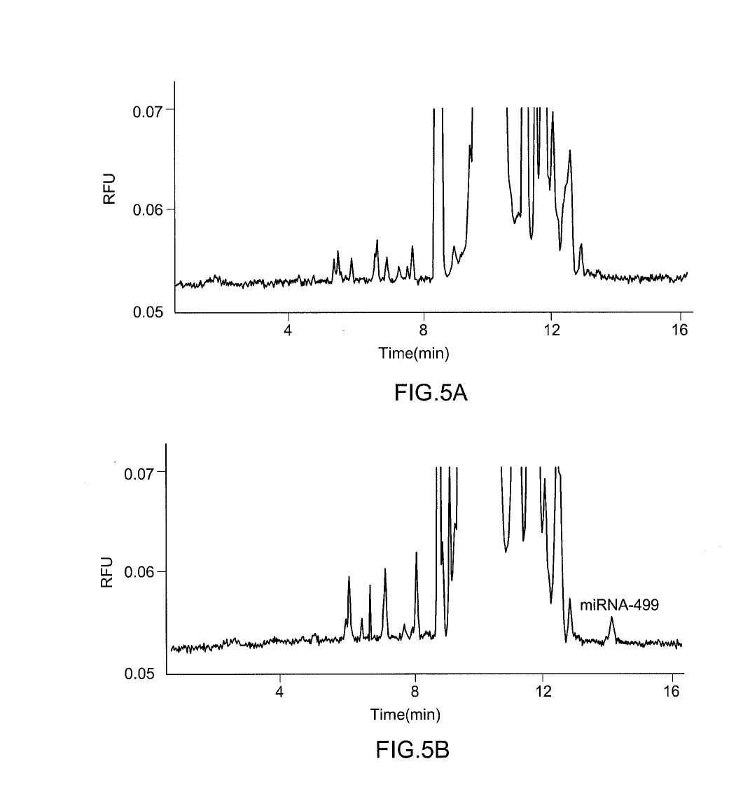

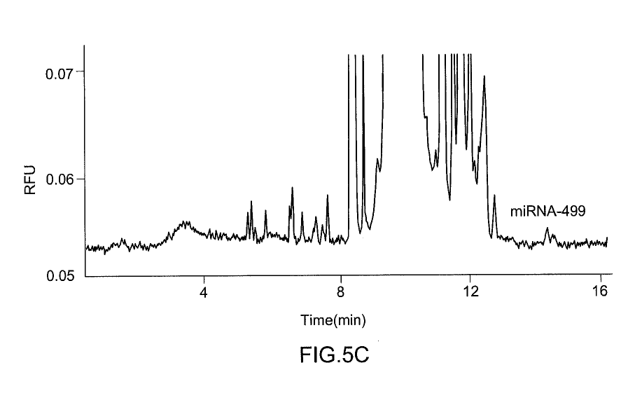

H9c2 cardiomyocytes (Korean Cell Line Bank) were cultured in DMEM containing 10% FBS and 1 vol % of penicillin-streptomycin. The medium was replaced every other day and the incubator used to culture the cells was maintained at 37° C. and 5% CO₂. Total RNAs were extracted from 1×10⁶ cells on a 100-mm dish using TRIzol and Triton X-100 with or without adding 50 fM miRNA-499. The extracted total RNAs and a DNA probe specific to miRNA-499 labeled with 6-FAM were hybridized at 40° C. for 15 minutes in TEN hybridization buffer after preincubation at 95° C. for 5 minutes, and then analyzed using a capillary electrophoresis with laser-induced fluorescence detector (CE-LIF). The CE system was PA 800 plus CE system (Beckman Coulter, Fullerton, Calif., USA) and the LIF detector was Beckman P/ACE System Laser Module 488 with excitation and emission wavelengths of 488 nm and 520 nm, respectively. A DNA-miRNA-499 complex was separated in 200 mM Tris-borate buffer (×10.0) by applying a voltage of 16 kV into an uncoated capillary (Beckman Coulter) having an inner diameter of 75 μm and a length of 40 cm. Sample injection was carried out at 0.5 psi for 50 seconds. The result is shown in FIGS. 5B and C. The peak of the DNA-miRNA-499 complex was observed from both cells with or without miRNA-499 added. This means that the miRNA present in a trace amount of 50 fM or less can be detected by CE/LIF. FIG. 5A shows the peak of the DNA probe specific to miRNA-499 labeled with 6-FAM.

The features and advantages of the present disclosure may be summarized as follows:

(i) The present invention provides a method for detecting a miRNA and a kit for detecting the same.

(ii) In accordance with the present invention, the miRNA present in a trace amount in a sample can be quantitatively analyzed in short time.

(iii) The detection method of the present invention may be used for fast diagnosis of various diseases wherein miRNAs are involved, for example, cardiovascular diseases including myocardial infarction.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A method for detecting a miRNA in a sample, comprising:
extracting an RNA from a sample to be analyzed;
hybridizing the extracted RNA with a single-stranded DNA labeled with a fluorescent material as a probe specific to a miRNA expected to be present in the sample in trace amounts of not more than 50 femtomolar in a hybridization buffer;
separating and identifying a DNA-miRNA complex using a capillary electrophoresis with laser-induced fluorescence detector (CE-LIF); and
identifying the presence of the miRNA in the sample by analyzing a peak of the DNA-miRNA complex.

2. The detection method according to claim 1, wherein the CE-LIF detector is one with an excitation wavelength of 400-500 nm and an emission wavelength of 500-600 nm.

3. The detection method according to claim 1, wherein the sample is a cell, serum, blood plasma, saliva, tear or urine.

4. The detection method according to claim 1, wherein the separation of the DNA-miRNA complex is performed using an uncoated capillary.

5. The detection method according to claim 4, wherein the uncoated capillary has an inner diameter of 50-100 μm and a length of 20-60 cm.

6. The detection method according to claim 4, wherein the separation is performed by applying a voltage of 10-20 kV into the uncoated capillary using Tris-borate buffer.

7. The detection method according to claim 3, wherein the cell is a cardiomyocyte.

8. The detection method according to claim 1, wherein the miRNA is one or more selected from the group consisting of miRNA-499, miRNA-1, miRNA-133 and miRNA-208.

9. The detection method according to claim 8, wherein the miRNA is one or more selected from the group consisting of miRNA-499 of SEQ. ID. NO. 1, miRNA-1 of SEQ. ID. NO. 2, miRNA-133a of SEQ. ID. NO. 3 and miRNA-208 of SEQ. ID. NO. 4.

10. The detection method according to claim 9, wherein the miRNA is miRNA-499 of SEQ. ID. NO. 1.

11. The detection method according to claim 1, wherein the probe is a probe of SEQ. ID. NO. 5.

12. The detection method according to claim 1, wherein the fluorescent material is 6-carboxyfluorescein.

13. The detection method according to claim 1, which further comprises determining a most suitable hybridization buffer by hybridizing the miRNA with a single-stranded DNA labeled with a fluorescent material as a probe specific to the miRNA in various hybridization buffers

14. The detection method according to claim 1, wherein the hybridization buffer is one or more selected from the group consisting of TNM buffer, PBS buffer, Tris-Cl buffer, SSC buffer, HEN buffer and TEN buffer.

15. The detection method according to claim 14, wherein the hybridization buffer is TEN buffer.

16. A kit for detecting a miRNA present in a trace amount of not more than 50 femtomolar, which is for use in a capillary electrophoresis with laser-induced fluorescence detector (CE-LIF), comprising: a probe that can be specifically hybridized with the miRNA and a fluorescent material that can be labeled at the probe, or a probe labeled with a fluorescent material that can be specifically hybridized with the miRNA; a hybridization buffer; and a buffer for separation of a DNA-miRNA complex.

17. The kit for detecting a miRNA according to claim 16, wherein the miRNA is one or more selected from the group consisting of miRNA-499, miRNA-1, miRNA-133 and miRNA-208.

18. The kit for detecting a miRNA according to claim 17, wherein the miRNA is one or more selected from the group consisting of miRNA-499 of SEQ. ID. NO. 1, miRNA-1 of SEQ. ID. NO. 2, miRNA-133a of SEQ. ID. NO. 3 and miRNA-208 of SEQ. ID. NO. 4.

19. The kit for detecting a miRNA according to claim 16, wherein the probe is a probe of SEQ. ID. NO. 5.

20. The kit for detecting a miRNA according to claim 16, wherein the hybridization buffer is one or more selected from the group consisting of TNM buffer, PBS buffer, Tris-Cl buffer, SSC buffer, HEN buffer and TEN buffer.

21. The kit for detecting a miRNA according to claim 16, wherein the separation buffer is Tris-borate buffer.

22. The kit for detecting a miRNA according to claim 16, wherein the fluorescent material is 6-carboxyfluorescein.


SEQUENCE LISTING (5 sequences)

SEQ ID NO: 1 - RNA, 21 nt, Homo sapiens
uuaagacuugcagugauguuu

SEQ ID NO: 2 - RNA, 22 nt, Homo sapiens
uggaauguaaagaaguguguau

SEQ ID NO: 3 - RNA, 22 nt, Homo sapiens
uuugguccccuucaaccagcug

SEQ ID NO: 4 - RNA, 18 nt, Homo sapiens
auaagacgagcaaaaagc

SEQ ID NO: 5 - DNA, 21 bp, Artificial Sequence
  miRNA-499 probe
aaacatcactgcaagtcttaa